(19) United States
(12) Patent Application Publication
(10) Pub. No.: US 2024/0293076 A1
(43) Pub. Date: Sep. 5, 2024

(54) EUSTACHIAN TUBE/TYMPANIC MEMBRANE ANALYSIS SYSTEM

(71) Applicant: Forrest P. Gauthier, Homestead, FL (US)

(72) Inventor: Forrest P. Gauthier, Homestead, FL (US)

(21) Appl. No.: 18/589,565

(22) Filed: Feb. 28, 2024

Related U.S. Application Data
(60) Provisional application No. 63/449,279, filed on Mar. 1, 2023.

(51) Int. Cl. A61B 5/00; A61B 5/12

(52) U.S. Cl. CPC A61B 5/48; A61B 5/126

(57) ABSTRACT

A device can be used to diagnose inner ear barotrauma, and train users how to properly equalize, preventing future inner ear barotrauma. It may also be used as a method of predicting near term equalization ability, thus predicting probable success in the user's ability to tolerate rapid pressure changes, whether they be caused by air travel, mountain driving, skin and scuba diving, or other causes of rapid barometric changes.

### CROSS-REFERENCE TO RELATED APPLICATIONS

This application claims the benefit of U.S. Provisional Application No. 63/449,279, filed Mar. 1, 2023, the disclosure of which is incorporated herein by reference.

### BACKGROUND OF THE INVENTION

This invention relates to a device, software and methods for measuring and processing human tympanic and Eustachian tube health by measuring and analyzing nasal cavity gas and sound pressure for tympanometry and pharyngotympanic measurements. The invention consists of the Sensor Probe, the design of the mobile phone App, a pair of sound drivers of the ‘earbud’ style, and methods of using the invention.

Individuals with compromised or inadequate tympanic membrane, middle ear, Eustachian tube and or Tensor veli palatini, Levator veli palatini and Salpingopharyngeus muscle function often have issues in air travel, travel to altitude, hiking at altitude, elevators in tall buildings, and other circumstances involving barometric pressure changes. The problems stem from barometric pressure changes associated with changes in altitude while the middle and or inner ear pressure is not changing at the same rate as the outer ear. Inner Ear Barotrauma (IEB) is also one of the most common Self Contained Underwater Breathing Apparatus (SCUBA) and skin diving injuries, caused by the failure or inability to properly equalize the pressure in the middle ear with the surrounding water pressure.

IEB can cause vertigo (temporary or chronic), hearing loss and tinnitus. The severity of injury can range from mild to severe. Symptoms may include dizziness, ear pain, bleeding from the nostrils, a feeling of fullness in the ears, ear drum injury, and temporary hearing loss. The best way to prevent IEB is keep the pressure in the middle and inner ear equal in the outer ear as the change in pressure is occurring. In air travel, this should be done as airplanes gain and lose altitude, especially during landing, while driving in mountainous regions or while going up and down in elevators. Scuba divers need to equalize their ear passages with the surrounding water pressure as soon as they start to descend.

The problem is worse when individuals are subject to allergies, have colds or other temporary illness that impacts the nasal passages and sinuses, active infections in the airways and sinuses, or have prior injury. Children have the same issues, and are often more prone to injury than adults. In part this may be due to the angular position of the Eustachian tube as an individual ages, migrating from 10° from horizontal in infants to about 35° from horizontal in adults.

IEB can be prevented by properly equalizing, which involves opening the Eustachian tube and the Tensor veli palatini, Levator veli palatini and Salpingopharyngeus muscles, allowing air pressure in the back of the throat to match with that in the middle ear space. There are six muscles that help the Eustachian tube open and close. The muscles are located in the ear, head, neck, soft palate, and jaw. Some individuals are able to accomplish this easily by yawning or swallowing, while others must make more significant efforts to succeed in this task. Because these muscles are rarely consciously exercised, when faced with the need to equalize individuals often do not know how to do so effectively.

The outer ear is made up of the auricle (the visible part of the ear) and the external auditory canal. The auricle helps to collect sound waves and direct them into the canal, where they reach the eardrum. The eardrum, or tympanic membrane, vibrates in response to sound waves and sends these vibrations through the middle ear.

The middle ear is a small, air-filled chamber that contains three small bones called the ossicles (the malleus, incus, and stapes). These bones amplify and transmit the vibrations from the eardrum to the inner ear. The middle ear also includes the Eustachian tube, which connects the middle ear to the back of the throat and helps to equalize pressure between the middle ear and the outside environment.

The inner ear is a complex structure that contains the cochlea, which is responsible for hearing, and the vestibular system, which is responsible for balance. The cochlea is a spiral-shaped organ that contains hair cells, which convert sound vibrations into electrical signals that are sent to the brain. The vestibular system includes the semicircular canals and the otolith organs, which detect changes in head position and movement.

Scuba diving can cause barotrauma and disease in the ear due to the rapid and high-pressure changes in pressure that occur during diving. Barotrauma occurs when the eardrum is stretched or ruptured by the pressure difference between the middle ear and the outside environment. This can cause pain, temporary or permanent hearing loss, and vertigo. Inner ear barotrauma can also occur when air bubbles form in the inner ear, which can cause vertigo, tinnitus, and hearing loss. To prevent barotrauma, divers should equalize the pressure in their ears by using the Valsalva maneuver or other methods before diving and throughout the descent.

Eustachian tube dysfunction (ETD) is a common condition that affects the middle ear and is characterized by a malfunctioning of the Eustachian tube, which connects the middle ear to the back of the throat. Inner ear barotrauma (IEB) is an injury to the inner ear caused by a pressure difference between the middle ear and the external environment. The relationship between ETD and IEB is complex and multifaceted, and the effect of ETD on IEB is not fully understood.

One of the most significant ways that ETD can contribute to IEB is by causing a build-up of negative pressure in the middle ear. The Eustachian tube is responsible for equalizing the pressure between the middle ear and the external environment. When the tube is not functioning properly, it can result in a negative pressure build-up in the middle ear, which can lead to IEB.

Additionally, ETD can also contribute to IEB by causing inflammation and swelling in the middle ear. This can further obstruct the Eustachian tube, making it even more difficult to equalize the pressure between the middle ear and the external environment. This can increase the likelihood of IEB occurring.

There are also some studies suggesting that ETD can lead to IEB by affecting the cilia in the middle ear. Cilia are tiny hair-like structures that help to move fluid and mucus out of the middle ear. When ETD causes inflammation and swelling in the middle ear, it can damage the cilia and lead to a build-up of fluid in the middle ear. This can further contribute to IEB by increasing the pressure in the middle ear. ETD can have a significant effect on IEB. ETD can lead to a build-up of negative pressure in the middle ear, inflammation and swelling in the middle ear, and damage to the cilia in the middle ear. All of these factors can contribute to the development of IEB. It is important for individuals who are experiencing symptoms of ETD to seek medical attention in order to prevent the development of IEB.

Studies have used acoustics to detect inner ear dysfunction or disease by measuring the ear's response to sound. These studies have primarily used two methods: otoacoustic emissions (OAEs) and auditory brainstem response (ABR) testing.

OAEs are low-level sounds that are emitted by the ear in response to a stimulus, such as a click or tone. These emissions can be measured using the prior art of a small probe that is inserted into the ear canal. Abnormal OAEs can indicate inner ear dysfunction, such as sensorineural hearing loss.

ABR testing measures the electrical activity of the ear in response to sound. This prior art test can be used to detect inner ear disorders such as nerve deafness and vestibular disorders.

Recent studies have shown that OAEs and ABR testing can be used effectively to detect inner ear dysfunction in both adults and children. For example, one study found that OAEs were able to detect inner ear dysfunction in 93% of adults with sensorineural hearing loss. Another study found that ABR testing was able to detect inner ear dysfunction in 100% of children with vestibular disorders.

Overall, these studies suggest that acoustics-based techniques such as OAEs and ABR testing can be a useful tool in detecting inner ear dysfunction or disease.

Studies have shown the muscles responsible for opening and closing the Eustachian tube at the base of the tube have an indirect yet characteristic sound when contracting. The muscles responsible are the Tensor veli palatini, the Levator veli palatini, and the Salpingopharyngeus.

The Tensor veli palatini is a muscle that originates from the medial pterygoid plate and the spine of the sphenoid bone, and inserts onto the cartilage of the Eustachian tube. This muscle is responsible for tensing the soft palate and opening the Eustachian tube.

The Levator veli palatini is a muscle that originates from the petrous portion of the temporal bone and inserts onto the cartilage of the Eustachian tube. This muscle is responsible for elevating the soft palate and opening the Eustachian tube.

The Salpingopharyngeus muscle is a muscle that originates from the cartilage of the Eustachian tube and inserts onto the pharyngeal wall. This muscle is responsible for opening the Eustachian tube.

In addition to the direct measurements of the Eustachian tube the deflection of the tympanic membrane, also known as the eardrum, can be used to detect dysfunction. The tympanic membrane is a thin and delicate membrane that separates the external ear from the middle ear. and plays a crucial role in the auditory system, as it vibrates in response to sound waves and transmits these vibrations to the ossicles of the middle ear, which in turn transmit the vibrations to the inner ear. The movement of the tympanic membrane can be measured and detected using various methods, which include:
Tympanometry: This is a non-invasive method that measures the movement of the tympanic membrane in response to changes in air pressure. A small probe is inserted into the ear canal, and a gentle pressure is applied to the ear. The probe then measures the changes in the pressure and the movement of the tympanic membrane, which can help to identify any problems with the middle ear, such as fluid buildup or a perforated eardrum.
Acoustic reflectometry: This method uses a small probe that is inserted into the ear canal, similar to tympanometry. However, instead of applying pressure, the probe emits a sound wave, and the reflections of the sound wave are measured to assess the movement of the tympanic membrane. This method can help to identify problems such as a blocked ear canal or a perforated eardrum.
Otoscopy: This is a visual examination of the tympanic membrane using an otoscope, which is a small instrument with a light and a magnifying lens. The otoscope is inserted into the ear canal, and the tympanic membrane can be visualized. This method is useful for identifying any abnormalities such as a perforated eardrum or any other structural abnormalities of the tympanic membrane.
Audiology Test: This method uses pure tone audiometry to measure the hearing threshold, which is the softest level of sound that a person can hear. This test helps to detect any sensorineural hearing loss, which is caused by damage to the inner ear or to the nerve pathways that connect the inner ear to the brain. Sensorineural hearing loss can be caused by a variety of factors, including exposure to loud noise, aging, and certain medical conditions.
Imaging Studies: Imaging studies such as CT or MRI can be used to visualize the tympanic membrane and the surrounding structures of the middle ear. These studies can help to identify structural abnormalities or any other problems with the middle ear.

Tympanometry and acoustic reflectometry are two important prior art diagnostic tools used to assess Eustachian tube dysfunction (ETD). These tests are non-invasive, safe and easy to perform, making them a valuable addition to the diagnostic armamentarium of an otolaryngologist.

Tympanometry is a test that measures the movement of the eardrum in response to changes in air pressure within the ear. The test is performed by inserting a small probe into the ear canal that applies a pressure change to the ear. The probe also measures the movement of the eardrum in response to this pressure change. The results are displayed on a graph called a tympanogram, which shows the eardrum's movement in relation to the air pressure applied.

Normal middle ear function is characterized by a symmetrical and shallow tympanogram with a peak pressure of around +200 daPa. ETD is diagnosed when the tympanogram is flat or negative. This indicates that the Eustachian tube is not functioning properly, preventing the middle ear pressure from being equalized.

Acoustic reflectometry is another test used to assess ETD. This test uses sound waves to measure the reflectivity of the eardrum. The test is performed by inserting a small probe into the ear canal that sends a sound wave through the ear. The probe then measures the reflection of the sound wave from the eardrum. The results are displayed on a graph called an acoustic reflectometry graph, which shows the reflectivity of the eardrum in relation to the sound wave.

Normal middle ear function is characterized by a high reflectivity of the eardrum. ETD is diagnosed when the reflectivity of the eardrum is low, indicating that the Eustachian tube is not functioning properly.

The Eustachian tube and tympanic membrane are important structures in the ear that play a crucial role in maintaining proper hearing and balance. ETD and tympanic membrane deflection (TMD) are common conditions that can lead to hearing loss and other ear-related symptoms. In order to accurately diagnose and treat these conditions, it is important to have accurate methods for measuring ETD and TMD.

One method that has been proposed for measuring ETD and TMD is to use increased pressure in the sinuses to assess the function of the Eustachian tube and tympanic membrane. This method is based on the principle that the Eustachian tube and tympanic membrane are connected to the sinuses through the nasopharynx, and that changes in pressure in the sinuses can affect the function of these structures.

One way to increase pressure in the sinuses is to use a device called a Valsalva maneuver. This is a technique in which the patient exhales forcefully against a closed airway, which raises the pressure in the sinuses. By measuring the deflection of the tympanic membrane and the opening and closing of the Eustachian tube during the Valsalva maneuver, researchers can assess the function of these structures.

Another method to increase pressure in the sinuses is to use a device called a pressure chamber. This device applies a controlled pressure to the sinuses, which can be used to assess the function of the Eustachian tube and tympanic membrane. Researchers can measure the deflection of the tympanic membrane and the opening and closing of the Eustachian tube during the pressure chamber test to assess the function of these structures.

Typical yet not exclusive or limited sinus target pressures range from 200 to 400 daPa while stimulating the tympanic membrane at 200 to 250 Hz at a pressure level of 85 db.

Overall, increasing pressure in the sinuses is a proven method for measuring ETD and TMD. The Valsalva maneuver and a pressure chamber combined is a method we are proposing for increasing pressure in the sinuses, Eustachian tube and middle ear for assessing the function of the Eustachian tube and therefore the proper deflection of the tympanic membrane.

### SUMMARY OF THE INVENTION

The proposed device can be used to diagnose IEB, and train users how to properly equalize, preventing future IEB. It may also be used as a method of predicting near term equalization ability, thus predicting probable success in the user's ability to tolerate rapid pressure changes, whether they be caused by air travel, mountain driving, skin and scuba diving, or other causes of rapid barometric changes.

Various aspects of this invention will become apparent to those skilled in the art from the following detailed description of the preferred embodiment, when read in light of the accompanying drawings.

### BRIEF DESCRIPTION OF THE DRAWINGS

FIG. 1 is a block diagram of a device to better diagnose ear, sinus, ETD and IEB conditions and dysfunction in accordance with this invention.

FIG. 2 is a side elevational view of the device of this invention.

FIG. 3 is an end elevational view of the device of this invention.

FIG. 4 is a side elevational view of the device of this invention.

FIG. 5 is a perspective view of the device of this invention.

FIG. 6 is a perspective view showing the device of this invention prior to use by a user.

FIG. 7 is a top plan view of a printed circuit board for use in the device of this invention.

FIG. 8 is a more detailed top plan view of the printed circuit board shown FIG. 7.

FIG. 9 is a bottom plan view of the printed circuit board shown FIGS. 7 and 8.

FIG. 10 is a block diagram of a system incorporating the device of this invention.

FIG. 11 is a block diagram of a flowchart illustrating a method of using device of this invention.

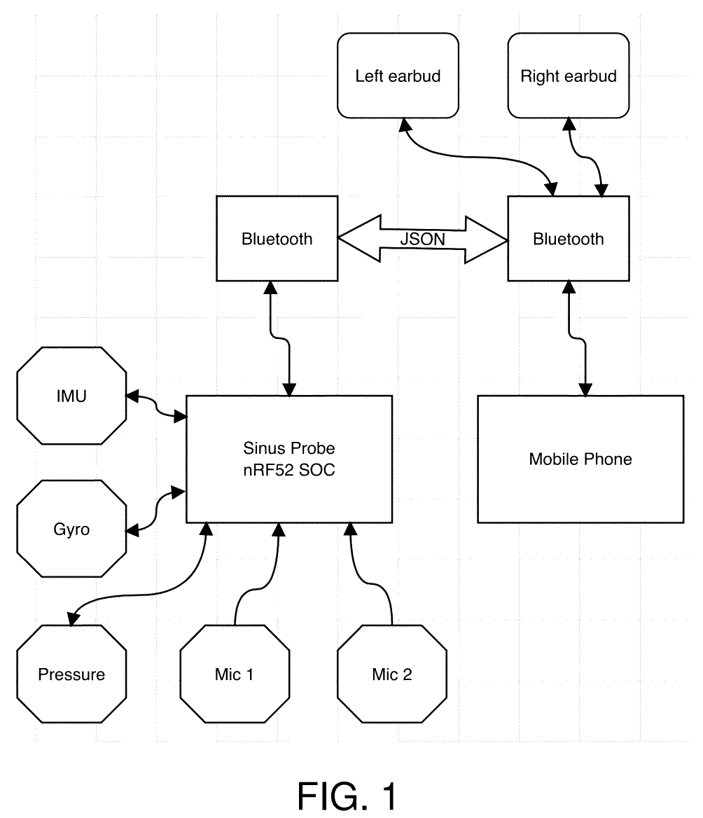

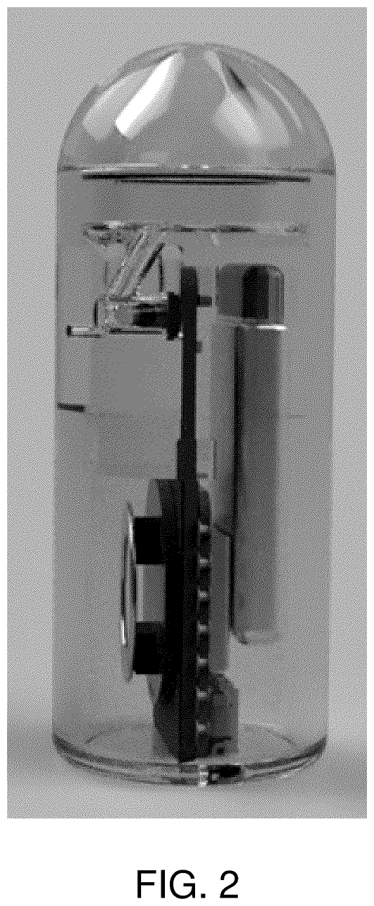

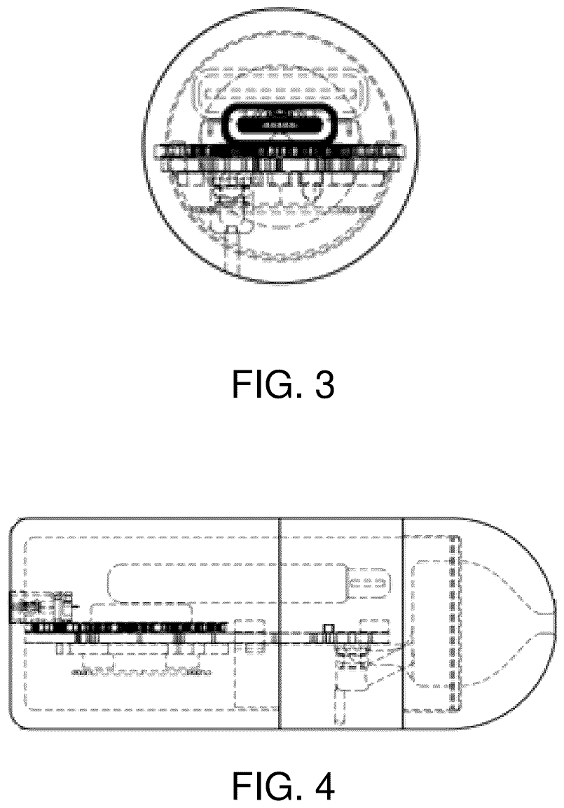

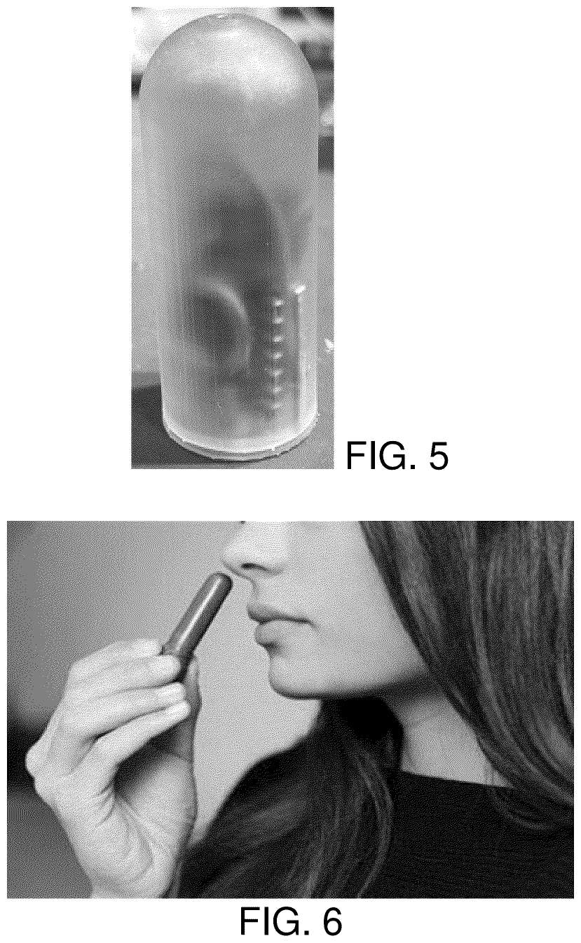

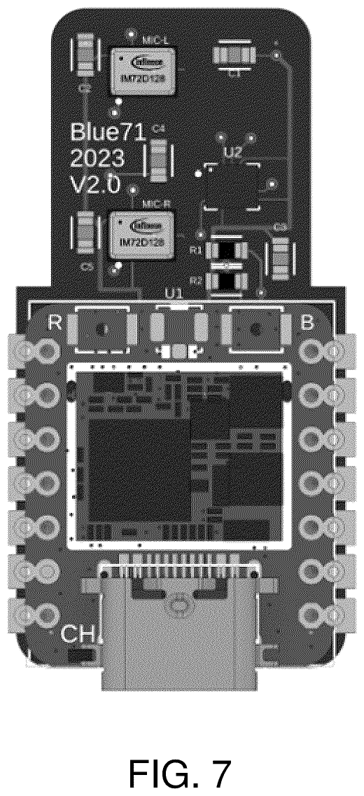

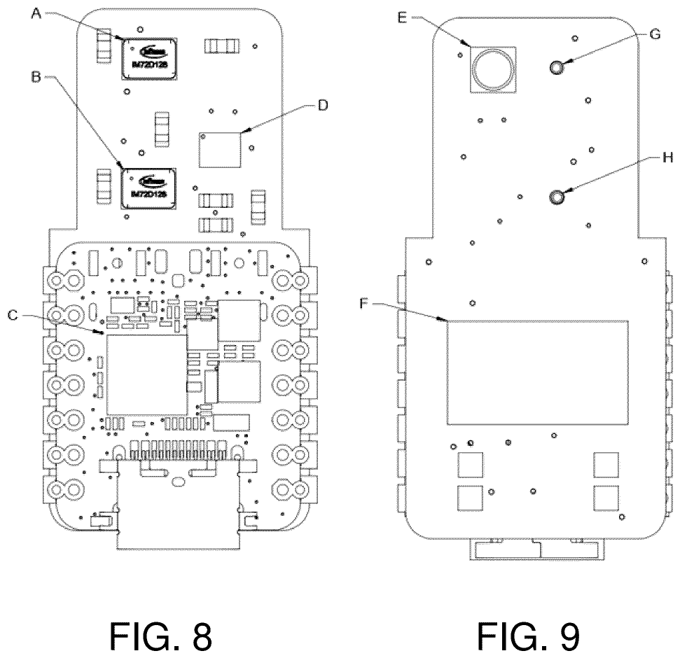

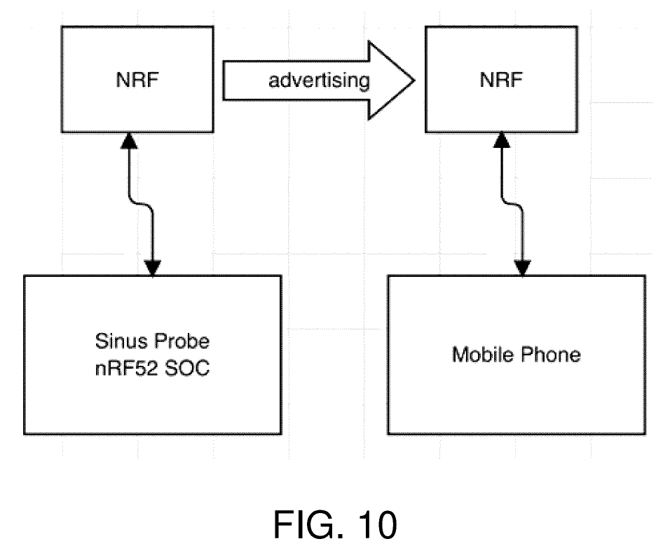

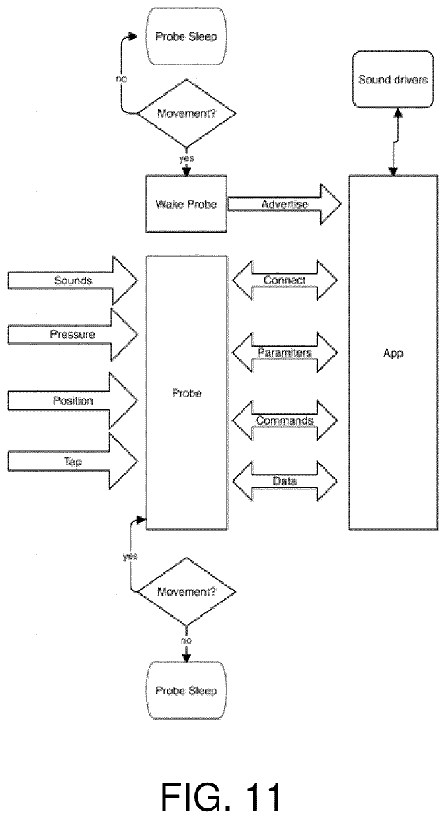

### DETAILED DESCRIPTION OF THE PREFERRED EMBODIMENT

The invention consists of a novel device and novel methods to better diagnose ear, sinus, ETD and IEB conditions and dysfunction offering significant improvements over the prior art.

The invention uses a new device and methods which are less intrusive, usable for self-diagnosis, and provides a method to exercise the Eustachian tube and the Tensor veli palatini, Levator veli palatini and Salpingopharyngeus muscles outside of a medical or other dedicated diagnostic facility. The invention incorporates a simple and low-cost device and the software embedded in the device referred to as the Sinus Probe and the software of the mobile App. The invention includes the design of the Sensor Probe, the software of the Sensor Probe, the software of the mobile App and the methods described to create the invention results.

The methods described may include the use of one or more of a standard “earbud” style sound drivers intended to be fluid coupled and approximately sealed to the outer ear canal. This sound driver device is of standard utility incorporating the prior art of such devices. No claim is made to the novelty of design of this device, however claim is made to the method of using this device to transmit a sound pressure wave into the ear canal to stimulate the tympanic membrane at a test frequency, amplitude and timing. This pressure wave is controlled in the specified method by the software to create one or more specific set of frequencies, and a one or more specific set of amplitudes in synchronization with the intent of the test being preformed

The methods described include a programmable mobile device of no specific make, design or model. This device is of standard utility incorporating the prior art of such devices. No claim is made to the novelty of design of this device, however claim is made to the method of using this device to provide the user interface to the Sensor Probe and to the specific proprietary software installed on the mobile device.

This description of the invention is the current reduction to practice and not intended to limit the use of other sensor or processing technology which may provide similar functions. The design of the Sensor Probe microelectronics, battery power and wireless charging of the battery enables the Sensor Probe to be compact, portable, biologically shielded, and fluid tolerant.

The Sinus Probe device consists of a plastic housing which encloses multiple gas passages, a printed circuit board, battery and battery charging inductance coil. In the current embodiment the housing comprises two chambers, one containing the electronics, battery and coil, the other containing the sample gas. The diameter of the current device is 22 mm, although other diameters could be used. The sample gas chamber is fluidly coupled and generally pressure sealed to the nostril orifice via a small 5 mm orifice in the upper end of the chamber at the apex of the curvature. The upper surface of the sample gas chamber is rounded, such that when it is held by the user against a nostril it provides an approximate sealing surface. The sample gas chamber is fluid coupled to the pressure sensor, temperature sensor and the first microphone by gas passages in the housing. An optional second microphone is fluid coupled to the ambient gas surrounding the sensor by a independent gas passage in the housing. The electronics chamber contains the microelectronics, power supply and charging circuits.

In the current embodiment the sample gas chamber and electronics chambers are isolated with fluid coupling ports from the gas chamber to the pressure sensor and the microphone. The sample gas chamber and electronics chambers could be fluidly connected, either openly or restricted with an optional hydrophobic membrane or similar barrier. In either case the pressure sensor, temperature sensor and first microphone are fluid coupled to the sample gas chamber. One or more flexible hydrophobic and or biologic barriers may be used in either chamber to reduce the presence of moisture from the sample gas.

The design and current enablement of the Sensor Probe device includes but is not limited to:
a Nordic Semiconductor nRF52840 System On Chip (SOC) Microcontroller Unit (MPU) to collect and process data from the various sensors and communicate to external devices by radio frequency. This SOC may also configure external devices using NFC-A radio frequency data transmission;
a LSM6DS3TR Inertial Measurement Units (IMU) is included to track the device's movement, orientation and to detect taping inputs from the user. Movement is typically yet not exclusively used to wake the device from power saving mode while orientation is typically used to determine the proper positioning of the device. Taping inputs are optionally used as user inputs to the software;
a first and optionally second MEMS MSM261D35 PDM microphone are used to capture audio input from the sinus cavity and optionally from the ambient noise around the sensor. In the current embodiment one microphone is fluid coupled by a port to the gas chamber and a second microphone is used to collect ambient sounds through a port to the outside of the device. The ambient microphone is used to reduce common mode sounds from the primary microphone; and
a TE MS5837-02BA or TE MS5837-30BA pressure sensor, which is used to measure the temperature, ambient pressure, device altitude and sinus gas pressure.

Other pressure sensors have also been used and the MS5837 is not meant to limit the sensor used in a particular embodiment.

To provide power to the invention, it is equipped with a 5 Watt Qi wireless battery charger and a 350 mAh Lipo battery. This battery and charger are typical sizes and capacity and are not intended to limit the use of other battery and charger technology or size. The wireless charger allows the device to be charged without the need for a physical connection, while the Lipo battery provides a long-lasting, rechargeable power source.

The Sinus Probe is optionally connected to one or more sound drivers of the “earbud” style using the Sinus Probe Bluetooth radio. Typically for the left and right ear using the Bluetooth version 5 radio incorporated with the nRF52840. This allows the invention to inject both pressure and frequency waves into the desired outer ear canal.

In typical operation the Sinus Probe is connected to the mobile device using the Bluetooth radio incorporated with the nRF52840, allowing the user to access, monitor and control the device remotely.

The Sinus Probe is optionally connected to a mobile device using the NFC-A radio incorporated with the nRF52840, allowing the user to collect configuration data from the device necessary for remote identification.

In the current embodiment a replaceable hydrophobic biological barrier membrane is installed between the gas chamber and the ports to the microphone and pressure sensor. This membrane is easily replaced between uses.

The Sinus Probe may optionally incorporate a hydrophobic membrane between the sample gas and electronics chambers.

The Sinus Probe may optionally incorporate a hydrophobic membrane between the nostril orifice and the sample gas chamber.

The Sinus Probe may optionally contain a secondary orifice in the sample gas chamber, which could optionally be used for cleaning and flushing the chamber.

The Sinus Probe may optionally incorporate a replaceable external hydrophobic membrane sleeve or similar barrier, used as a biological and moisture barrier.

The Sinus Probe may optionally include a method of isolating the electronics components from possible intrusion of liquids, aerosols, or particulates by encapsulation or coatings and therefore use only a single chamber for sample gas and electronics.

One method of the invention involves injecting a pressure and frequency signal into the ear canal using sound drivers which are approximately sealed to the outer ear canal. While stimulating the tympanic membrane with a typical 200-250 Hz constant or sweep tone at typically 85 Bd SPL and simultaneously capturing the frequency and pressure in the sinus cavity. Both frequency and pressure signals are recorded simultaneously to ensure accurate comparison. The actual and difference signals of the injected and captured signals is then processed and analyzed in terms of time, frequency and amplitude, this providing insights to the movement of the tympanic membrane, the harmonic dampening of the membrane and the middle ear's response to the stimulus.

Another method of the present invention involves injecting a pressure and frequency signal into the ear canal using ear buds which are approximately sealed to the outer ear canal while simultaneously capturing the frequency and pressure in the sinus cavity while the patent is preforming a Valsalva maneuver and or performing a swallowing maneuver and or other muscular maneuvers intended to open the Eustachian tubes and the Tensor veli palatini, Levator veli palatini and Salpingopharyngeus muscles. Both frequency and pressure signals are recorded with simultaneously to ensure accurate comparison. The difference between the injected and captured signals is then processed and analyzed in terms of time, frequency and amplitude, providing measurements of the tympanic membrane and inner ear's response to the stimulus.

Another method of the present invention is to provide a visual representation of the sinus pressure on the display of the mobile device in order to train the patient in the proper form and pressure of the Valsalva maneuver, visually and tactically indicating the typical target of 200 to 500 daPa (0.29 to 0.73 psia) of pressure to obtain in the sinus cavity while performing the maneuver. In the current embodiment this display is in the form of a graph, however it is anticipated the sinus pressure value will also be used for a game play training program to assist in training the user in the proper execution of the Valsalva maneuver.

Another method of the invention is the recording and processing of sounds created by OAE. OAEs, which are low-level sounds, such as a click or tone, that are emitted by the Tensor veli palatini, the Levator veli palatini, and the Salpingopharyngeus in response to a pressure, sound or muscular stimulus. The sounds are then processed to diagnose the efficacy of the Valsalva maneuver or other maneuvers.

The above listed emissions are measured by the microphone in the Sinus Probe without the need or use of external audio recording or stimulus in the outer ear canal.

The Sinus Probe is an embedded system custom sensor device with a PCB which includes a nRF52840 SOC, a first and second PDM microphones, a pressure sensor and a six axis inertia unit and associated parts. The BLE radio of the nRF52 is used to connect to the proprietary iOS App installed on the mobile phone.

A fourth sensor on the PCB is a LSM6D3TR IMU. The IMU functions in this project are all internal modes of the IMU, specifically the “wake up” and “significant movement” interrupt functions are both set by internal registers in the IMU. The gyroscope XYZ threshold values and the interrupt are also set-up in the IMU registers.

The Nordic nRF52840 SOC is the main processing unit and the Bluetooth radio for the system. The code for this project is developed using VSCode with the platformIO environment and the Nordic mRF connect, SDK and other appropriate libraries. The code is written in C++. The Zephyr RTOS is used as the operating system.

In use, the First PDM microphone collects data as frequency and pressure levels in the sinus cavity, while the Second PDM microphone collects background ambient noise. On BLE activation, both microphones collect data alternately on the rising and falling edge of the PDM clock signal. The PDM values from the microphones are processed and filtered by the nRF52 PDM unit then stored as pulse code modulated (PCM) values into a buffer as alternating sequential 16 bit values. At the conclusion of the collection of a set of values in the buffer, the nRF52 generates an interrupt then immediately begins to store the uninterrupted PCM data from the two microphones into a second buffer.

On selection by the Boolean in the software an optional noise reduction function in the code compares the adjacent PCM 16 bit values from the first microphone and the second microphone and computes the differential of the two values (active noise reduction).

On selection by the software Boolean an optional a notch filter may be applied to the PCM values to reduce the amplitude of a specific frequency in the PCM stream.

The value of the first microphone is processed using a modified fast Fourier transform to extract the amplitude of a center frequency and bandwidth of 20 frequencies. The resultant 20 amplitude values are stored in an array of float values using the index of the two PCM buffers from which the values are from. The 20 float amplitude values stored in the selected array are converted to a JSON string then transmitted to the iOS App a using Bluetooth and the appropriate write characteristic.

On completion of the processing, the address of the first buffer is rewritten to the PDM unit for use as the next buffer.

On each interrupt of the PDM Unit the function which processes the data alternates between the two buffers used by the PDM unit and the alternate array of float values. The FFT functions run at sufficient speed to process each buffer in less time than the PDM unit fills the alternate buffer. This process continues uninterrupted until the PDM unit is deactivated by the start Boolean from the start BLE characteristic. In the current implementation the FFT spectrum analyzer, noise filter and notch filter are processed in the Sinus Probe software using the DSP library from the nRF Connect SDK.

The LSM6D3TR IMU is used to detect when to wake up or put to sleep the system, to detect a double tap to initiate actions, and to show the orientation of the sensor device.

The IMU internal registers are programmed to detect the “significant motion” event as specified in the LSM6DTR data sheet using values stored in the parameter structure. At the end of the Sinus Probe use, the IMU and system is put to sleep to conserve the battery. When the user picks up the device the IMU detects this “significant motion” which creates and interrupt to the nRF52, waking it from low power sleep.

The accelerometer is also used to detect a double tap action, this is set up using the internal machinery of the IMU as specified in the data sheet and the appropriate settings of the registers. A double tap, detection transmits the appropriate BLE notify to the iOS app

During operation, the gyroscope is used to indicate the orientation and position of the sensor device. Threshold values are set in the registers of the IMU from values in the parameter structure. When the threshold values are met the IMU generates an interrupt, the registers are read, and the XYZ values are converted to a JSON string and sent to the iOS App using the appropriate BLE characteristics.

While the sensor device is being handled the significant motion threshold is regularly being triggered, creating interrupts, which reset the low power sleep timers. When the sensor device is set down the significant motion threshold is not exceeded, the interrupts stop and the low power sleep timer expires putting the entire system into low power mode.

The MS5837 pressure sensor is used to detect the pressure of the users sinus cavity. The pressure sensor is activated on the Boolean value in the start struct. It is calibrated using the value of the iOS device pressure sensor which is stored in the parameter structure. This calibration value is transmitted from the iOS device to the Sinus Probe each time the device connects to the central Bluetooth device. The pressure sensor and its companion temperature are read on a periodic basis based upon a timer in the Sinus Probe code. On the interval the pressure and temperature of this sensor is sent to the iOS App through the appropriate BLE characteristic.

### Sensor Device Operation States

On wake from systemOff, the mpu goes to boot-up.
After boot check the battery, if bat volts>bat.min init the sensors.
The BLE characteristics are initialized.
Begin to advertise.
On BLE connection load the param JSON then parse into the pram struct.
The MS pressure sensor is calibrated to zero using the MS calibration value from the iOS.
The sensors are initialized and the timers are started as called for in the start struct.
If the test type is 1 process the pressure and temp.
If the test type is 2 process the spectrum analyzer and pressure.
If the test type is 3 process spectrum analyzer and pressure
When the start bool goes false, stop the PDM DNA and complete the processing of existing buffers.
If significant motion is not detected during the time interval, disconnect the BLE, sleep the sensors and go to systemOff.

### Details of the Use and Programming of the Sensors

### LSM6D3TR IMU

The accelerometer of the IMU is used to control the low-power modes of the sensor based upon significant movement of the device and control the data collection using double tap detection.
The gyroscope of the IMU is used to shown the orientation of the device and guide the user.
In SystemON operation the Accelerometer and Gyroscope are set to normal power mode.
Prior to SystemOFF mode the Gyroscope is placed power-down mode.
Prior to SystemOFF mode the Accelerometer is placed into low power-mode.
If the IMU has not detected “significant motion” as defined int the IMU registers for a value of time contained in the parameter structure put the sensors into low power and the nRF52 into SystemOFF mode.
The no-motion minutes to sleep variable is in the param JSON.
On detection of significant motion wake the entire system using the IMU interrupt.
On waking up the system is in cold boot and begins BLE advertising.
On BLE connect the iPhone will write the param JSON to the Probe.
While the system is awake if the gyroscope XYZ orientation values exceed the threshold value an interrupt is asserted. The sensor then pushes the XYZ values to the central using the gyroscope notify BLE characteristic.
The two thresholds of significant movement are set by the imuThreshold and imuTime parameter values.
The IMU enables the double tap feature. On tap detection notify the central using the notify BLE characteristic.
When in sleep mode the microphones, pressure sensor, and MPU core sleep are all put into low power mode.
The double tap register thresholds are #defines.
The IMU is connected using SPI and has two interrupts. The IMU libraries take care of the HAL layer switching between i2c and SPI. The interrupt system uses the single interrupt of with an option to use the second interrupt.

### MS5837 Pressure Sensor

The pressure sensor is operated using the MS5837 i2c-1 bus.
The two sensor types, 02 bar or 30 bar are be selected by the msType key/value in the param JSON.
The sensor interrupt timer duration is a milliseconds value of the msInterval key/value in the param JSON.
The sample resolution of the device is be set by the msResolution key/value in the param.
The i2c bus is driven at 400 kHz.
The timer to service the pressure sensor is interrupt driven to enable precise time based slope calculations.
The msCalibration value in the param is used to calibrate the pressure sensor to the ambient pressure. Pressure readings from the sensor are adjusted to zero using this calibration value.
The result of the pressure (mbar) and temperature (Celsius) reading is placed in a float struct and sent by a read/notify BLE characteristic using pressure.mbar and pressuretempc keys.

### PDM Microphones

The PD and microphones are controlled using the pulse density modulation interface.
The PDM edge register is set by the pdmEdge key/value pair in the param.
The PDM sample frequency register is set by the pdmFreq key/value pair in the param JSON.
The PDM sample size is set by the pdmsize key/value pair in the param JSON.
The system has a first and second PDM microphones. The first microphone will be left microphone the second microphone is right.
Which microphone is left or right is set by pdmEdge key/value.
The microphone L/R gain is set by the pdmLgain and pdmRgain key/value pairs in the param JSON.
The PDM unit is started and stopped by the value of the pdmStart key/value in the param JSON.
Two or more PDM buffers are created depending on the observed processing time. The pointer to the sequential buffer is written on interrupt of the PDM unit.
The PDM unit is set to operate in stereo mode using a single set of buffers.
Microphone right is used for common mode noise reduction of microphone left. When recording microphone right is used to subtract common the mode signal from microphone left.
The noise reduction feature is turned on and off by the bool in the start struct.
When noise reduction is turned off the PDM unit remains in stereo mode and the alternate sample is indeterminate and unused.
A notch filter is defined using the notchFreq and notchWidth.

### PDM Notch Filter

The notch filter is a narrowband filter to reduce the amplitude of the specified frequency at the specified bandwidth.
If activated the notch filter is enabled. On PDM Unit interrupt the PCM value is read then the adjacent PC value is read. We then apply the notch filter to both values, and rewrite the results back to the original locations.

### PDM Noise Filter

The noise filter is an active background noise eliminator, which inverts the 2nd microphone value from the 1st microphone value.
If activated the active noise filter is enabled. On PDM Unit interrupt apply the noise filter function to the two adjacent PCM values and then rewrite the differential to the location of the 1st microphone.

### PDM Buffer Processing

If the spectrum analyzer is enabled, the function reads the First microphone value and applies the fast Fourier transform to that value, and returns the result to the struct.
The output of the audio buffer processing is a multi-band spectrum analyzer of frequency and amplitude.
The spectrum analyzer has a common bandwidth value of spectrumWidth and a low and high center frequency range of lowFreq and highFreq. The 20 center frequency channels are filed in with the inclusive division of values between the low and high values.
The amplitude processing of the spectrum analyzer uses the DSP functions of the nRF connect SDK and may also use a Goertzel algorithm.
The size of each buffer contains the number of audio samples for each rendering of the spectrum analyzer.
The single bandwidth and multiple center frequencies are defined in an array of key/value pairs in the param.spectrumArray BLE characteristic.
The processing of a buffer and the creation of the output is dependent upon whether the microphones are on or off and the interrupts from the PDM unit.
The amplitude of each frequency band will be placed into a struct then formatted for transmission using the spectrumArray JSON format and a notify/read BLE characteristic.
If the noise filter is enabled, common mode noise is removed prior notch filter function.
If a notch filter is defined the filter is applied before the spectrum analyzer function.

### Battery Power

The unit is powered by a rechargeable lithium ion battery with a wireless Qi battery charger.
The battery analog voltage is read using an analog read of pin A0 the applying this formula—voltage*(3.3/4095.0);
The battery voltage is processed using an interval timer of a #define number of milliseconds.
The resulting float value is set using the bat Volts key and a read BLE characteristic.
When the unit is placed on the wireless charger, significant motion should stop, and the unit will be put into systemOff mode.

### Data Structures Between the Sinus Probe and the Mobile Device

| • | JSON, parameters, BLE Characteristic central to peripheral write. | | |
|---|---|---|---|
| • | uint_t | msType = 30; | // type of MS sensor int enum 02 or 30 |
| • | uint_t | mBeforeSleep = 300; | //the number of minutes of no motion |
| | before sleep. | | |
| • | uint_t | imuThreshold = 254; | // IMU acceleration threshold to wake |

| | up enum 0-254 | | |
|---|---|---|---|
| • | uint_t | imuTime = 62; | // shifted to 6 bits - the duration value for the |
| | IMU wakeup enum 0-62 | | |
| • | uint_t | msInterval = 3000; | // time in MS of the MS sensor readings. |

| • | uint_t | msResolution = 4096,; | // ms sensor ADC resolution enum 1024, |
|---|---|---|---|
| | 2048, 4095, 8192. | | |
| • | float | msCalibration = 1024.00; | // barometric pressure from the iPhone |

| | | | |
|---|---|---|---|
| • | float | pdmFreq = 44000; | // PDM microphone sample frequency |

| | | | |
|---|---|---|---|
| • | bool | pdmEdge = 1; | // PDM sample edge polarity enum 0,1. |

| • | uint_t | pdmTime = 250; | // PDM sample size in milliseconds |
|---|---|---|---|
| • | uint_t | pdmLgain = 40; | // PDM gain left enum 0-80 with 40 the default |
| • | uint_t | pdmRgain = 40; | // PDM gain right enum 0-80 with 40 the |
| | default | | |
| • | float | notch Width = 5.50 | //notch filter bandwidth. |

| • | JSON, msSensor, BLE Characteristic peripheral to central notify. | |
|---|---|---|
| • | float | mbar = 1028.00 // compensated pressure from MS sensor |
| • | float | tempc = 31.12 // compensated temp from MS sensor |

| • | JSON, imu, BLE Characteristic peripheral to central notify (notify payload | | |
|---|---|---|---|
| | is > 32 bytes) | | |
| • | int16_t | x = 65534; | // gyro angle two's compliment. |
| • | int16_t | y = 65534; | //gyro angle two's compliment. |
| • | int16_t | z = 65534; | // gyro angle two's compliment |

| • | JSON, start, BLE Characteristic central to peripheral write. | | |
|---|---|---|---|
| • | int_t | testType = 0; | // test type number 0-3 “None, Valsalva, Eustachian, |
| | Tympanic” | | |
| • | bool | start = 0; | // start the test.. |
| • | bool | pdm = 0; | //start the microphones. |
| • | bool | ms = 0; | //start the pressure sensor. |
| • | bool | notch = 0; | //start the notch filter |
| • | bool | noise = 0; | //start of the noise filter |

| • | float | freqTone = 220.50;// earbud injection tone frequency, doppler | |
|---|---|---|---|
| | display center point, noise filter center | | |
| • | float | freqWidth = 10.50; | // the frequency bandwidth of the of the |
| | spectrum analyzer notch | | |
| • | float | freqLow = 150.00; | // the spectrum analyzer array low frequency |

| | value | | |
|---|---|---|---|
| • | float | freqHigh = 8000.00; | // the spectrum analyzer array high |

| | frequency value | | |
|---|---|---|---|
| • | JSON, bat, BLE Characteristic central to peripheral read. | | |
| • | float | batVolts = 3.85; | // 1.2f - pre-computed float of Probe battery |

volts.

| • | JSON, tap, BLE Characteristic peripheral to central notify. | | |
|---|---|---|---|
| • | bool | IMUTap=0; | //the double tap detection of the IMU |

| • | JSON, fftValues, BLE Characteristic peripheral to central write. | | |
|---|---|---|---|
| • | int_t[20] | amplFFT = 0; | // the amplitude of each frequency from the |

FFT calculations.

### The Design of the Mobile Application Software

In the current embodiment the look and feel of the mobile device App is roughly patterned after other Blue71 and Pneuma mobile device applications. In the current embodiment the App does not process the raw audio data, the processing of the audio is done in the Sensor Probe and the results are sent to the App over Bluetooth in JSON key/value pairs. This simplifies the App to be primarily the user interface and control functions. The Sensor Probe is controlled using a single start/stop button in the App. A second screen is used to enter the parameter settings using simple well known SwiftUI pickers. The phone connects to the Sensor Probe and to a set of sound drivers of the earbud style using Bluetooth and eight Bluetooth characteristics.

The App opens with the splash screen. The splash screen has a single central button labeled connect. On activation of the connect button the App scans for the Sensor Probe advertising filtered by the service characteristic of the Probe. If the Probe is found the App connects to it. If no Probe is found a message to “wake up the Probe or move Probe into range” is displayed. On successful Bluetooth connection, the barometric sensor of the mobile device is read and sent to the Probe using the parameter JSON based upon the default values. Read the battery voltage and compare it to a minimum voltage value stored as a constant, if less display a pop with “Probe battery too low” and disconnect from the Probe, if greater send the parameter JSON to the Probe using the appropriate Bluetooth characteristic. Set the Probe in Valsalva mode. Then connect the App to the earbuds and transmit a 1 second tone to the earphones to validate the connection. If the earphone connection fails use a default error message. The mode JSON string is sent to the Probe using the appropriate BLE characteristic. Start a timer to check the Probe and earbud battery voltage every three minutes.

On the top of the first page in the Title area left to right is an SF Symbols “airpods” and a SF Symbol “battery . . . ” for AirPod battery voltage. Then the screen Title. On the right a SF Symbols icon “sensor” for the Probe then the same SF Symbol battery icon for Probe battery voltage. The device icons change state as they are connected to the phone by Bluetooth, gray for disconnected. The series of battery icons change according the battery voltages and are updated about every three minutes.

Below the line separator in the V stack is an H stack with four view objects.

The first object from the left margin across ⅔ of the screen width is the spectrum analyzer bar chart. This bar chart has 20 bars across the horizontal laid on top of an XY grid of thin, light-gray graph lines. The graph lines and bar charts are enclosed in a light-gray line on the top bottom left and right of the chart. Below the bottom of the chart and centered on each bar is the frequency assigned to that bar, which is displayed vertically with one place right of the decimal.

The next view in the horizontal stack is a single bar chart with no graph lines and enclosed in matching, thin, light-gray borders. This bar chart occupies ⅙ of the screen width. Below the border is a legend for the bar chart labeled “Pressure”. This bar chart occupies the same vertical space as the spectrum analyzer view.

Next in the horizontal stack occupying the remaining ⅙ of the screen width are two horizontal views each using half of the height of the spectrum analyzer chart. In the top half is a USDZ 3-D object of the Probe which is rotated about the center of its axis according to the gyroscope XYZ values. Below the 3-D object is a circular graph displaying the temperature with a legend below the graph marked “Temp”.

Below the graphs in the vertical stack is a horizontal stack of eight buttons, indicators and pickers. On the left are the SF Symbols icon for Play and Pause indicating the Start and Stop states of the Probe. Next is a series of pickers which display the legend and the value selected. The first is the “Mode”, next is “Low”, next is “High”, next is “Width”, next is “Notch”, next is “Vol” , next is “Tone”. After the pickers is the SF symbols icon “slider.horizontal.3”.

The Start/Stop button sends the entire “start” JSON string to the Probe after setting the state of the “start” bool. On start the start bool is set to true, on stop the start bool is set to false.

The Probe has double tap detection from the inertia unit in the Probe. If the Probe detects a double tap this is sent the App using a Bluetooth characteristic notify. This notification is equivalent to activation of the start, stop button and must be reflected in the start button of the app.

The boot-up values of the bools and the values of the pickers of the Start struct is:
testType=0;
start=0;
pdm=0;
ms=0;
notch=0;
noise=0;
freqTone=0;
freqWidth=0;
freqLow=0;
FreqHigh=0;

The Mode picker selects “Valsalva, Eustachian, Tympanic’. Like all the pickers the resulting pick is shown on the screen and saved as a Mode-indexed-variable to be restored to the pickers when the matching mode is selected. On any change of the Mode picker the system goes to Stop and sends the stop JSON to the Probe with the start bool set to false. The bool default is Valsalva.

When Valsalva is selected the Start struct is:
testType=1;
start=0;
pdm=0;
ms=1;
notch=0;
noise=0;
freqTone=0;
freqWidth=0;
freqLow=0;
freqHigh=0;

The Probe will then respond by sending the values for the pressure and temperature which are then shown on the appropriate charts. No spectrum analyzer data is received or displayed.

When Eustachian is selected the start JSON default is:

| • testType = 2; | |
|---|---|
| • start = 0; | |
| • pdm = 1; | |
| • ms = 1; | |
| • notch = ?; | // use the param value. |
| • noise = ?; | // use the param value. |

• freqTone = 0;
• freqWidth = 20.00;
• freqLow = 100.00;
• freqHigh = 8000.00;

Any change made to the pickers is stored in Mode-indexed-variable to be restored when the Eustachian mode is selected again. The Probe will then respond by sending the values for the FFT bar charts, pressure and temperature which are all shown on the appropriate charts.

When Tympanic is selected the start JSON default is:

| • testType = 3; | |
|---|---|
| • start = 0; | |
| • pdm = 1; | |
| • ms = 1; | |
| • notch = ?; | // use the param value. |
| • noise = ?; | // use the param value. |

• freqTone = 200.00;
• freqWidth = 5.00;
• freqLow = 100.00;
• freqHigh = 300.00;

On Start the tone frequency is sent to the airPods/earbuds, first the right side for 10 seconds, then the left side for 10 seconds and repeat until Stop. Any change made to the pickers is stored in Mode-indexed-variable to be restored when the Tympanic mode is selected again. The Probe will then respond by sending the values for the FFT bar charts, pressure and temperature which are all shown on the appropriate charts. The prototype tone code will be provided by the client.

The Low picker selects the low end of the frequency span of the spectrum analyzer. The default value and range of the picker may be modified by the Mode selected. Once a new value is selected by the picker that value is written back to the mode-index-variable to be used the next time that Mode is selected.

The High picker selects the high end of the frequency span of the spectrum analyzer. The default value and range of the picker may be modified by the Mode selected. Once a new value is selected by the picker that value is written back to the mode-index-variable to be used the next time that Mode is selected.

The Spectrum analyzer bar chart uses low frequency and high frequency as the bounds for the 20 bar charts. The first bar chart and the last bar chart are the values of high and low. The bar charts between are equal divisions of the remainder.

The Width picker selects the bandwidth range of the spectrum analyzer. The default value and range of the picker may be modified by the Mode selected. Once a new value is selected by the picker that value is written back to the mode-index-variable to be used the next time that Mode is selected.

The Vol picker selects the audio tone volume of the tone generator. The default is 40. Once a new value is selected by the picker that value is written back to the mode-index-variable to be used the next time that Mode is selected.

The Tone picker selects the audio tone frequency of the tone generator. The default is 150.00. Once a new value is selected by the picker that value is written back to the mode-index-variable to be used the next time that Mode is selected. This value is also used by the tone generator to create the tone for the earbuds. On start the tone generator transmits the tone to the left earbud for 10 seconds, then to the right earbud for 10 seconds, then repeats until the stop button is pressed.

The Edit button stops the system and navigates to the edit screen.

The spectrum analyzer bar graph receives its data from the Probe. Each value contained in the JSON string of FFT values is assigned to the bar chart from left to right. Each time the Probe writes new data to the App the bar chart is updated. The low frequency and high frequency values are divided inclusively into frequency steps. The value of the steps become the legend of the individual bars. When the values of low frequency and high frequency are changed the legends of the bars are changed accordingly. The scale of the bar charts is set by a constant value.

The pressure bar graph and the temp bar graph displays the values sent to the App from the MS JSON string. The pressure bar graph does not have a displayed vertical scale. In the code we want two values of threshold which changes the bar graph from green to yellow to red. We will enter the two thresholds and the bar chart scale as constant values in the app.

The temperature bar graph displays the temperature in a circular graph from 0 to 50 c (no legends) with a digital representation of the temperature in the center of the graph.

A 3D object of the Probe will be provided by the client. In the space above the temperature graph show the 3D object centered. The Probe will send XYZ values which will be applied to rotate the 3D object, indicating the actual orientation of the Probe in the 3D image in the app.

The Edit screen.

At the top of the screen on the left is the SF Symbols icon “chart.bar.xaxis” which returns to the main screen. In the center is the screen title.

Next in the v stack the edit screen is a simple vertical stack of text entry fields and pickers with labels for each field. The text entry fields match the fields of the parameter struct. On touch of the TextEdit field, the entry method will pop up, allowing the editing of the field according to the data type parameters. On completion of entry, using enter or save, write the value to the parameter structure. The values of the parameter structure are sticky and on close of the application the values remain for the next execution of the app. On update of any value write the parameter JSON to the Probe using the appropriate BLE characteristic.

The labels and text entry fields are as follows:

| • | MS sensor type: | Picker number 02 or 30. |
|---|---|---|
| • | Minutes before sleep | Picker number from 1 to 10. |
| • | Wakeup threshold | Picker number from 1 to 254. |
| • | Wakeup Time | Picker number from 0-62. |
| • | MS sensor Interval | Picker number from 100 to 1000 in hundreds. |
| • | MS sensor Resolution | Picker number of 1024, 2048, 4095, |
| | 8192. | |
| • | PDM freq | Number pad with decimal. |
| • | PDM edge | Picker number of 0 or 1. |
| • | PDM time ms | Number pad no decimal. |
| • | PDM L gain | Picker number between 0-80 with 40 in |
| | the center | |
| • | PDM R gain | Picker number between 0-80 with 40 in |
| | the center | |
| • | Notch filter Width | Number pad with decimal |

### Data Structures

### JSON, Parameters, BLE Characteristic Central to Peripheral Write

| • | uint_t | msType = 30; | // type of MS sensor int enum 02 or 30 |
|---|---|---|---|
| • | uint_t | mBeforeSleep = 300; | //the number of minutes of no motion |
| | before sleep. | | |
| • | uint_t | imuThreshold = 254; | // IMU acceleration threshold to wake |
| | up enum 0-254 | | |
| • | uint_t | imuTime = 62; | // shifted to 6 bits - the duration value for |

| | the IMU wakeup enum 0-62 | | |
|---|---|---|---|
| • | uint_t | msInterval = 3000; | // time in MS of the MS sensor readings. |

| • | uint_t | msResolution = 4096,; | // ms sensor ADC resolution enum 1024, |
|---|---|---|---|
| | 2048, 4095, 8192. | | |
| • | float | msCalibration = 1024.00; | // barometric pressure from the iPhone |

| | | | |
|---|---|---|---|
| • | float | pdmFreq = 44000; | // PDM microphone sample frequency |

| | | | |
|---|---|---|---|
| • | bool | pdmEdge = 1; | // PDM sample edge polarity enum 0,1. |

| | | | |
|---|---|---|---|
| • | uint_t | pdmTime = 250; | // PDM sample size in miliseconds |

| • | uint_t | pdmLgain = 40; | // PDM gain left enum 0-80 with 40 the |
|---|---|---|---|
| | default *see notes | | |
| • | uint_t | pdmRgain = 40; | //PDM gain right enum 0-80 with 40 the |

| | default *see notes | | |
|---|---|---|---|
| • | float | notch Width = 5.50 | //notch filter bandwidth. |

### JSON, msSensor, BLE Characteristic Peripheral to Central Notify

float mbar=1028.00;//compensated pressure from MS sensor
float tempc=31.12//compensated temp from MS sensor

### JSON, imu, BLE Characteristic Peripheral to Central Notify (Notify Payload is >32 Bytes)

| | |
|---|---|
| • int16_t x = 65534; | // gyro angle two's compliment. |
| • int16_t y = 65534; | //gyro angle two's compliment. |
| • int16_t z = 65534; | // gyro angle two's compliment |

### JSON, start, BLE Characteristic Central to Peripheral Write

| | | | |
|---|---|---|---|
| • | int_t | testType = 0; | // test type number 0-3 “None, Valsalva, Eustachian, |

| | Tympanic” | | |
|---|---|---|---|
| • | bool | start = 0; | // start the test.. |

| | | | |
|---|---|---|---|
| • | bool | pdm = 0; | //start the microphones. |

| | | | |
|---|---|---|---|
| • | bool | ms = 0; | //start the pressure sensor. |

| | | | |
|---|---|---|---|
| • | bool | notch = 0; | //start the notch filter |
| • | bool | noise = 0; | //start of the noise filter |

| • | float | freqTone = 220.50;// earbud injection tone frequency, doppler | |
|---|---|---|---|
| | display center point, noise filter center | | |
| • | float | freqWidth = 10.50; | // the frequency bandwidth of the of the |
| | spectrum analyzer notch | | |
| • | float | freqLow = 150.00; | // the spectrum analyzer array low frequency |
| | value | | |
| • | float | freqHigh = 8000.00; | // the spectrum analyzer array high |
| | frequency value | | |

### JSON, bat, BLE Characteristic Central to Peripheral Read

| | |
|---|---|
| • | float batVolts = 3.85; // 1.2f - pre-computed float of Probe battery |
| | volts. |

### JSON, tap, BLE Characteristic Peripheral to Central Notify

bool IMUTap;//the double tap detection of the IM

### JSON, fftValues, BLE Characteristic Peripheral to Central Write

int_t[20] amplFFT=0;//the amplitude of each frequency from the FFT calculations

In conclusion, an aspect of this invention is that the device places a pressure sensor and a microphone fluid coupled to the nasal orifice while approximately pressure sealing the orifice, fluidly connecting the nasal cavity, Eustachian tube, and tympanic membrane. This enables simultaneous recoding and or processing of gas pressure, sound pressure and sound frequencies from the nasal cavity, Eustachian tube and the middle and outer ear. This is novel in that it places and pressure sensor and a microphone in the nasal orifice fluid coupled to the nasal cavity.

Another aspect of this invention is that the device enables the simultaneous sound and pressure stimulation of the anterior of the tympanic membrane from the outer ear canal using a fluid coupled standard outer ear sound driver while recoding and or processing the gas pressure, sound pressure and sound frequencies inside the nasal cavity. This is novel and simpler than prior art tympanometry measurement systems which place a specialized recording microphone fluid coupled to the anterior of the tympanic membrane.

Another aspect of this invention is the processing of the sound and pressures signatures of the middle ear, tympanic membrane, Eustachian tube and nasal cavity to determine the potential of success of the user's ability to achieve middle ear equalization during activity likely to induce a ambient pressure change.

Another aspect of this invention is a non-invasive, self-administration method to pre-determine the potential for success of middle ear pressure equalization for users with compromised, diseased or impaired Eustachian tube, inner ear or the Tensor veli palatini, Levator veli palatini and Salpingopharyngeus muscles prior to an intended barosensitive activity.

Another aspect of this invention is the enablement of using ordinary earbud style sound drivers to inject the diagnostic tones into the outer ear canal, eliminating the need for specialized sound driver devices and the pathogen risk of shared devices.

Another aspect of this invention is the use of a Bluetooth connection to a personal mobile device with a custom application program to enable self-administration of the testing procedure and logging of the users instant or serial testing and results.

Another aspect of this invention is the gamified application program on the personal mobile device which enables a training and exercise method to enable the user to clear and stretch the Eustachian tube and or exercise the Tensor veli palatini, Levator veli palatini and Salpingopharyngeus muscles resulting in greater efficacy of middle ear equalization during activity likely to induce a ambient pressure change.

The principle and mode of operation of this invention have been explained and illustrated in its preferred embodiment. However, it must be understood that this invention may be practiced otherwise than as specifically explained and illustrated without departing from its spirit or scope.

What is claimed is:

1. A device for diagnosing inner ear barotrauma and for training users how to properly equalize and thereby prevent future inner ear barotrauma substantially as described and illustrated herein.
